(19) United States
(12) Patent Application Publication
(10) Pub. No.: US 2026/0256903 A1
(43) Pub. Date: Sep. 3, 2026

(54) DENGUE VACCINE

(71) Applicant: VLP Therapeutics, Inc., Wilmington, DE (US)

(72) Inventor: Wataru AKAHATA, Kensington, MD (US)

(73) Assignee: VLP Therapeutics, Inc., Wilmington, DE (US)

(21) Appl. No.: 19/553,925

(22) Filed: Mar. 2, 2026

Related U.S. Application Data
(60) Provisional application No. 63/765,955, filed on Mar. 3, 2025.

(51) Int. Cl. A61K 39/12; A61K 39/00; A61P 31/14; C07K 14/005; C07K 16/116; C12N 7/00

(52) U.S. Cl. CPC A61K 39/12; A61P 31/14; C07K 14/005; C07K 16/116; C12N 7/00; A61K 2039/5258; A61K 2039/575; C07K 2317/14; C12N 2770/24122; C12N 2770/24123; C12N 2770/24134; C12N 2770/24151

(57) ABSTRACT

Provided is a dengue virus like particle specific for type 4 dengue virus (DENV4 VLP), comprising a chimeric dengue structural protein, and a composition or vaccine comprising thereof, its use in the prevention or treatment of dengue infection. The chimeric dengue structural protein comprises prM and envelope regions and is a chimera of parts each derived from DENV1, DENV3 and DENV4.

### CROSS REFERENCE TO RELATED APPLICATIONS

This application claims the benefits of U.S. Provisional Patent Application No. 63/765,955 filed on Mar. 3, 2025. The entire disclosures of the prior application are hereby incorporated by reference.

### INCORPORATION BY REFERENCE OF SEQUENCE LISTING

The content of the electronically submitted sequence listing, file name: Q317893_sequence listing as filed; size: 14,255 bytes; and date of creation: Mar. 2, 2026, filed herewith, is incorporated herein by reference in its entirety.

### TECHNICAL FIELD

The present disclosure relates to a dengue vaccine.

### BACKGROUND ART

Dengue is a mosquito-borne viral disease that infects nearly 400 million people worldwide each year, and in severe cases causes dengue hemorrhagic fever (DHF), which is responsible for approximately 10,000 deaths each year. Dengue virus (DENV) is spread by both Aedes aegypti and Aedes albopictus mosquitos, which are endemic to more than 100 countries in all 6 habitable continents. Furthermore, in the coming decades, the range of both vector hosts will likely increase due to climate change, further increasing the population at risk of infection. No therapeutic drug to treat dengue infection is currently licensed, which makes an effective vaccine essential to meeting this global health threat.

A significant obstacle in the development of a dengue vaccine is the phenomenon known as antibody-dependent enhancement (ADE). There are four closely related, but antigenically distinct, DENV serotypes (DENV1-4). An individual's first DENV infection is typically mild or asymptomatic. However, a subsequent infection with a different DENV serotype may result in more severe disease due to ADE. ADE occurs when pre-existing, but non-neutralizing, anti-DENV antibodies against one DENV serotype form a DENV-antibody immunocomplex which enhances cellular entry into Fc-receptor-bearing cells and worsens the infection rather than preventing it. Though no clear clinical evidence has emerged yet, there is concern that an imbalanced vaccine could generate ADE, causing a person's first dengue exposure to lead to more severe illness. Therefore, to prevent ADE risk, any dengue vaccine must produce strong neutralizing antibody (NAb) responses against all four serotypes simultaneously.

The dengue virus (DENV) belongs to the family Flaviviridae, genus Flavivirus, and species Dengue virus. DENV has a positive-sense, single-stranded RNA genome that encodes three structural proteins, namely capsid (C), precursor membrane (prM/M), and envelope (E), as well as seven non-structural (NS) proteins, namely NS1, NS2A, NS2B, NS3, NS4A, NS4B, and NS5. These viral proteins are translated as a single polyprotein precursor, which is subsequently processed into individual mature proteins.

The viral particle is surrounded by a host-derived lipid bilayer originating from the endoplasmic reticulum (ER), into which the E and M proteins are embedded. Viral entry into host cells is initiated by receptor binding mediated by the E protein, followed by endocytosis. Upon exposure to the acidic environment of the endosome, the E protein undergoes conformational changes that promote fusion between the viral membrane and the endosomal membrane, thereby releasing the nucleocapsid into the cytoplasm.

The nucleocapsid comprises multiple copies of the capsid protein enclosing the viral RNA genome. Following uncoating, the viral RNA serves as a template for translation by the host cell machinery. The newly synthesized NS proteins assemble into a replication complex within vesicle packets derived from the ER membrane. Viral RNA replication proceeds within these membrane-associated replication compartments.

Following synthesis of progeny viral RNA, the RNA is packaged by the capsid protein, and immature virions bud into the ER lumen, thereby acquiring the host-derived lipid bilayer together with the E and prM proteins. During transport through the Golgi apparatus, prM is cleaved by the host protease furin to generate the mature M protein. This proteolytic processing results in maturation of the virion, which is then released from the host cell to initiate subsequent rounds of infection.

Mature DENV virions display an organized surface architecture consisting of 180 copies each of the E and M proteins. The E protein is arranged as homodimers on the viral surface and is composed of three domains, referred to as envelope domain I (EDI), envelope domain II (EDII), and envelope domain III (EDIII). Among these, EDIII is known to contain key neutralizing epitopes involved in antibody recognition. The schematic illustration of the Dengue genome is shown in FIG. 1.

To date, two dengue vaccines have been licensed and approved. The first one, Dengvaxia, is a live-attenuated tetravalent vaccine developed by Sanofi Pasteur and approved in 2016. However, long-term safety and efficacy studies revealed variable efficacy based on age and dengue sero-status at the time of vaccination, as children without prior dengue exposure at initial vaccination exhibited an elevated risk of severe dengue infection and hospitalization relative to children in the control group. Because of this risk, Dengvaxia is only recommended for persons 9-16 years of age in the areas of high dengue-endemicity (>70% seroprevalence). The second approved vaccine, Qdenga (TAK-003), is a live-attenuated tetravalent vaccine developed by Takeda. Because of the lack of data to assess the risk of enhanced disease in seronegative vaccinated children following DENV3 and 4 infections, the Strategic Advisory Group of Experts on Immunization (SAGE) strongly recommends further post-marketing studies to determine efficacy-risk profile in seronegative persons and recommended use of Qdenga only for children ages 6-16 in areas of high dengue disease burden. Therefore, developing an advanced dengue vaccine remains a major global health objective.

Virus-like particles (VLPs) are self-assembling structures composed of viral structural proteins without genomic DNA or RNA. VLP vaccines present a repetitive, high-density antigen profile that closely mimics the morphology of an authentic virus, making them highly immunogenic. However, the absence of the genomic information required for replication makes them relatively safe even in young children and immunocompromised individuals. The ability to immunize young children has critical importance for reducing dengue morbidity as a study of DHF prevalence in Thailand demonstrated that infants <1 year old represent a significant proportion of severe DHF cases. One hypothesis for the cause of severe dengue infection in infants is that it stems from ADE caused by maternal DENV antibodies acquired in utero. As the passively transferred maternal antibody titer wanes, the once protective antibody can result in enhanced dengue infection in the infant. While infants can receive live vaccines, there is concern for a live-attenuated dengue vaccine that the presence of the maternal antibodies may negatively affect the replication of one or more attenuated serotypes, leading to an imbalanced response. Immunocompromised individuals are another group at high risk of severe dengue infection; however, live-attenuated vaccines are not recommended due to the risk of increased replication or genetic reversion. VLP vaccines, on the other hand, are safe for immunocompromised persons because they lack the genetic material required for replication and reversion. These characteristics make a VLP vaccine attractive for protecting both high-risk groups (Thoresen et al., Journal of Virology 98 (5), 2024, e00239-24., the contents of this document are herein incorporated by reference).

### CITATION LIST

### Patent Literature

### [PTL 1]

U.S. Pat. No. 9,969,986

### [PTL 2]

U.S. Pat. No. 10,098,943

### [PTL 3]

U.S. Pat. No. 10,385,101

### Non Patent Literature

### [NPL 1]

Thoresen et al., Journal of Virology 98 (5), 2024, e00239-24.

### [NPL 2]

Haidar Ali et al., Journal of Clinical Virology Plus, Volume 5, Issue 3, 2025, 100227

### SUMMARY OF INVENTION

The present disclosure relates to a novel dengue vaccine.

In a first aspect, the present disclosure provides a dengue virus like particle specific for type 4 dengue virus (DENV4 VLP) comprising a chimeric dengue virus viral structural protein, wherein said chimeric dengue virus viral structural protein comprises prM and envelope regions and is a chimera of parts each derived from DENV1, DENV3 and DENV4.

In a second aspect, the present disclosure provides a nucleic acid molecule comprising a nucleotide sequence that encodes the chimeric dengue virus viral structural protein contained in the DENV4 VLP provided in the first aspect of the present disclosure.

In a third aspect, the present disclosure provides a composition or vaccine comprising the DENV4 VLP provided in the first aspect and/or the nucleic acid molecule provided in the second aspect.

In a fourth aspect, the present disclosure provides a method of producing an antibody or anti-serum comprising a neutralizing antibody, comprising contacting the DENV4 VLP provided in the first aspect of the present disclosure and/or the nucleic acid molecule provided in the second aspect of the present disclosure to a mammal.

In a fifth aspect, the present disclosure provides a method of treating or preventing dengue infection or a method of inducing and/or enhancing immune response against a dengue in a mammalian subject, comprising administering the composition provided in the third aspect of the present disclosure to the subject.

In a sixth aspect, the present disclosure provides a method of producing the DENV4 VLP provided in the first aspect of the present disclosure, comprising: culturing a cell which is transfected with a gene encoding the at least one dengue virus viral structural proteins contained in the DENV4 VLP; and recovering virus like particle from the cell culture. This aspect may further comprise the step of preparing a gene comprising a nucleotide sequence encoding the at least one dengue virus viral structural proteins contained in the virus like particle.

In a seventh aspect, the present disclosure provides a cell line expressing the DENV4 VLP of the first aspect.

In an eighth aspect, the present disclosure provides an improved method for producing dengue virus like particles.

### BRIEF DESCRIPTION OF DRAWINGS

FIG. 1 shows the schematic illustration of the wild type Dengue genome.

FIG. 2 shows the schematic design of old and new DENV4 VLP expression constructs. The “old” DENV4 VLP is disclosed in U.S. Pat. No. 10,098,943 as DENV4 VLP. Abbreviations:
SS=capsid signal sequence
prM=precursor membrane
EDI, II and III=envelope domains I, II and III
ST=stem
TM=transmembrane domain

FIG. 3 shows clonal expansion of DENV4 VLP-expressing stable cell lines resulted in robust release of the DNEV4 VLP into the supernatant.

FIG. 4 shows DENV4 VLP immunogenicity conferred by immunization with 1 μg old DENV4 VLP (transiently-expressed) vs 1 μg new DENV4 VLP (stably-expressed DENV4 VLP)

### DETAILED DESCRIPTION

### (1) a DENV4 VLP Comprising One or More Dengue Structural Proteins

In a first aspect, the present disclosure provides a virus like particle specific for type dengue virus (DENV4 VLP) comprising a chimeric dengue virus viral structural protein, wherein said chimeric dengue virus viral structural protein comprises prM and envelope regions and is a chimera of parts each derived from DENV1, DENV3 and DENV4.

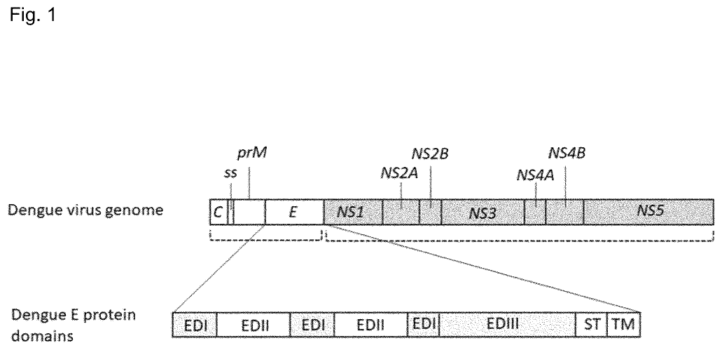

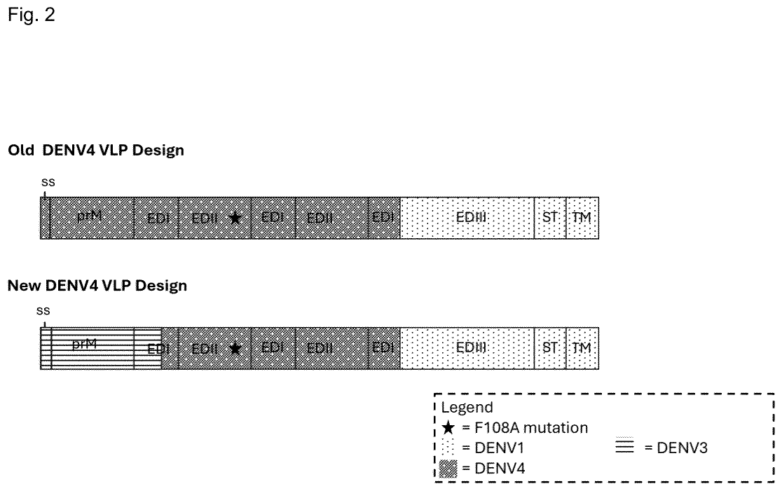

As shown in FIG. 1, Dengue virus viral structural protein consists of capsid protein, precursor membrane protein and envelope protein. The upper panel of FIG. 2 shows an “old” DENV4 VLP which was disclosed in U.S. Pat. No. 10,098,943. The lower panel of FIG. 2 shows a representative embodiment of the DENV4 VLP of this disclosure indicated as “new”.

In this embodiment, the DENV4 VLP comprises at least one of those structural proteins and preferably, a precursor membrane protein (prM) and an envelope protein. The Dengue virus viral structural protein may further comprise one or more regions corresponding to the initiation codon and a signal sequence to the amino terminal of the prM sequence.

The amino acid sequence of the chimeric Dengue virus viral structural protein constituting the DENV4 VLP is composed of amino acid sequences of corresponding region of naturally occurring DENV1, DENV3, and DENV4 viral structural proteins. The whole DENV4 VLP comprises regions of DENV1, DENV3 and DENV4.

Viral structural proteins of dengue virus types 1~4 have been identified and available at various public databases such as GenBank database. For example, Dengue virus type 1 (WestPac strain): Accession No. U88535, Dengue virus type 2 (S1 vaccine strain): Accession No. M19197, Dengue virus type 3 (Singapore 8120/95 strain): Accession No. AY766104 and Dengue virus type 4 (ThD4_0476_97 strain): Accession No. Y618988 and Dengue virus type 4 (Dominica strain): Accession No. M14931.

Each amino acid parts derived from the viral structural proteins of DENV1, DENV3, and DENV4 that constitute the DENV4 VLP of this disclosure contains more than 30 amino acids and exhibits at least 70%, 75%, 80%, 85%, 90%, 95%, or 98% identity with the corresponding region of the naturally occurring DENV1, DENV3, or DENV4 viral structural proteins, including the prM and envelope proteins. In one embodiment, the modified protein is a mutant of a chimeric dengue virus viral structural protein in which no more than 10% of the amino acids are deleted, substituted, or added to the full-length chimeric viral structural protein, including the prM and envelope regions composed of the corresponding regions of naturally occurring DENV1, DENV3, and DENV4. “Corresponding region” and “sequence identity” can be determined using bioinformatics programs for sequence alignment and analysis, such as BLAST, BESTFIT, GAP, and PILEUP/PRETTYBOX. The Sequence Analysis Software Package from the Genetics Computer Group at the University of Wisconsin Biotechnology Center (1710 University Avenue, Madison, WI 53705) may be employed.

As shown in FIG. 1, Dengue virus viral structural proteins comprises prM and E regions and E region comprises EDI, EDII, EDIII stem and transmembrane domains. In the DENV4 VLP of the present disclosure, the full length of each region or domain may be composed of a fragment of one type of DENV, or of two or more parts of different types of DENVs. In addition, signal sequence may preferably be derived from DENV1, DENV3 or DENV4. In the envelope region of the viral structural protein constituting the DENV4 VLP, amino acid Phe at position 108 of naturally occurring Dengue virus envelope protein is preferably altered to Ala (F108A).

According to the present disclosure, one or more chimeric dengue structural proteins or fragments thereof discussed above may be used as long as they spontaneously assemble into a particulate structure. For example, when eukaryotic cells expressing a gene encoding prM and envelope proteins of the chimeric dengue structural proteins or fragments thereof are cultured, the proteins are generated by the cells and assemble to give VLPs, and the VLPs can be collected from the cell culture supernatant.

The present disclosure addresses one or more of the above needs by providing the DENV4 VLPs, vectors encoding the VLPs, and antibodies (and antibody-like molecules including aptamers and peptides) that specifically bind to DENV4 antigen, together with the uses thereof (either alone or in combination) in the prevention or treatment of dengue infections.

As used in the specification and claims, the term “antibody” refers to a molecule which is capable of binding to an epitope or antigenic determinant. The term covers a whole antibody and an antigen-binding fragment thereof, including a single-chain antibody. Such antibodies may include human antigen binding antibody fragments and include, but are not limited to, Fab, Fab′ and F(ab′) 2, Fd, single-chain Fvs (scFv), single-chain antibodies, disulfide-linked Fvs (sdFv) and fragments comprising either a VL or VH domain. The antibodies can be from any animal origin including birds and mammals. Preferably, the antibodies are mammalian e.g., human, murine, rabbit, goat, guinea pig, camel, horse and the like, or other suitable animals e.g., chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598, the disclosure of which is incorporated herein by reference in its entirety.

The antibody recognizes a type 4 dengue structural protein. The dengue structural protein may be a naturally occurring protein or modified protein of the naturally occurring protein or a fragment of the naturally occurring protein or the modified peptide. The modified protein may be a fragment of the naturally occurring viral structural protein.

In one embodiment, the modified protein derived from a type 4 dengue structural protein has at least 70%, 75%, 80%, 85%, 90%, 95% or 98% amino acid sequence identity to the naturally occurring protein. In one embodiment, the modified protein derived from a dengue is a mutant where at most 10% of the amino acids are deleted, substituted, and/or added based on the fragment of naturally occurring viral structural protein of the dengue virus.

In one embodiment, the present disclosure provides a DENV4 VLP comprising a chimeric dengue virus viral structural protein having an amino acid sequence represented by SEQ ID No: 1.

SEQ ID NO: 1 contains the following regions: initiation codon: M (laa), signal sequence (2-15aa), pr sequence (16-106aa), M sequence (107-181aa), and Envelope region (182-676aa).

The present disclosure also provides a DENV4 VLP having at least 70%, 75%, 80%, 85%, 90%, 95% or 98% amino acid sequence identity to SEQ ID NO. 1.

### (2) Nucleotide and Vector

In the second aspect, the present disclosure provides a nucleic acid molecule comprising or consisting of a nucleotide sequence encoding the DENV4 VLP as provided in the first aspect of the present invention.

In one embodiment, the present disclosure provides a nucleic acid molecule comprising a nucleotide sequence that encodes a dengue virus viral structural protein that provide the DENV4 VLP as described above.

In one embodiment, the present disclosure provides an expression vector comprising the nucleic acid molecule as described above, wherein the vector optionally comprises an expression control sequence operably linked to the nucleic acid molecule.

Examples of expression control sequences include, but are not limited to, promoter such as CMV promoter, phage lambda PL promoter, the E. coli lac, phoA and tac promoters, the SV40 early and late promoters, and promoters of retroviral LTRs.

### (3) Composition or Vaccine

In the third aspect, the present disclosure provides a composition or vaccine comprising DENV4 VLP provided in the first aspect of the present disclosure and/or the nucleic acid molecule provided in the second aspect of the present disclosure.

In one embodiment, the present disclosure provides a composition comprising the DENV4 VLP as described above or the nucleic acid molecule as described above.

The composition may further comprise a pharmaceutically acceptable carrier and/or adjuvant.

As used herein, the term “adjuvant” is meant a compound that, when used in combination with a specific immunogen in a formulation, will augment, alter or modify the resultant immune response. In certain embodiments, the adjuvant is used in combination with the DENV4 VLP. Modification of the immune response includes intensification or broadening the specificity of either or both antibody and cellular immune responses.

Common adjuvants include aluminum containing adjuvants that include a suspension of minerals (or mineral salts, such as aluminum hydroxide, aluminum phosphate, aluminum hydroxyphosphate) onto which antigen is adsorbed. In other embodiments, the adjuvant is an aluminum-free adjuvant. The adjuvant can include one or more immunostimulatory components. The immunostimulatory component can include an oil and water emulsion, a liposome, liposaccharides, a lipopolysaccharide, a saponin, and an oligonucleotide.

The term “combination” used herein means two or more active ingredients are administered to a patient simultaneously in the form of a single entity or dosage, or those active ingredients are administered to a patient as separate entities either simultaneously or sequentially with no specific time limits, wherein such administration provides therapeutically effective levels of the two components in the body, preferably at the same time.

In one embodiment, the composition or vaccine is a tetravalent composition that includes DENV VLPs specific for DENV1, DENV2 and DENV3 respectively in addition to DENV4 VLP of the present disclosure. Known DENV1 VLP, DENV2 VLP and DENV3 VLP may be employed in this embodiment, for example DENV VLPs are disclosed in U.S. Pat. No. 10,098,943 and Thoresen et al., Journal of Virology 98 (5), 2024, e00239-24, the contents of these disclosures are herein incorporated by reference.

Namely, the present disclosure provides a composition comprising DENV1 VLP, DENV2 VLP, DENV3 VLP and DENV4 VLP, isolated nucleic acid molecules, each of which encodes the dengue viral structural proteins contained in one of the VLPs, and/or vectors comprising the nucleic acid molecules, which optionally include an expression control sequence operably linked to the nucleic acid molecule.

### (4) Method of Producing an Antibody

In the fourth aspect, the present disclosure provides a method of producing an antibody against a dengue or an antiserum containing a neutralizing antibody against a dengue, comprising contacting the DENV4 VLP, the nucleic acid molecule or the expression vector comprising the nucleic acid molecule discussed above to a mammal.

The antibody produced in this aspect may be used for passive immunization against a dengue virus-causing pathogen in a mammal by administering the same to the mammal, and thus prevent the mammal from dengue virus infection or treat a disease or condition caused by dengue infection in the mammal.

The antibody produced in the fourth aspect of the present disclosure may be humanized using a conventional technique. Thus, in one embodiment, the method provided in the fourth aspect of the disclosure further comprises a step of humanizing a non-human mammal produced antibody. The antibody or humanized antibody provided by this aspect may be used for preventing a human subject from dengue virus infection or for treating a disease or condition caused by dengue infection in the subject.

The antibody produced according to this aspect may be used in vitro to select a subpopulation from immune cells such as B-cell and T-cell derived from the patient, which are then re-administered to the patient.

Antiserum can be obtained by the conventional manner. Blood samples are taken from the immunized non-human animal, and the blood is processed so as to obtain the antiserum, i.e., the antibody-containing liquid component of the blood. The non-human mammal is preferably selected from the group consisting of rat, mouse, hamster, pig, rabbit, horse, donkey, goat, sheep, guinea pig, lama, and non-human primate such as chimp.

(5) Method of Treating a Disease Caused by a Dengue Infection in a Subject or Preventing a Subject from Dengue Infection

In the fifth aspect, a method of treating a disease or condition caused by dengue infection such as dengue fever in a subject, wherein the DENV4 VLP provided in the first aspect, the nucleotide molecule or the vector comprising the nucleotide molecule provided in the second aspect, or the composition provided in the third aspect is administered to the subject. By administering the above listed VLP, nucleotide molecule or vector or composition to the subject, immune response against a dengue can be enhanced and thus, the disease or condition caused by the dengue infection can effectively be treated. In this aspect, the DENV4 VLP, nucleotide molecule or vector, or composition may be administered to the patient locally to the affected organ or systemically.

A method of preventing a subject from a dengue infection or from a disease caused by a dengue infection, comprising administering the DENV4 VLP of the first aspect, the nucleotide molecule or vector provided in the second aspect or the composition of the third aspect to the subject in need thereof.

According to the present disclosure, the virus like particle can also be applied for immune therapy. The DENV4 VLP may be applied ex vivo to cells derived from the patient or a human cell line which are subsequently administered to the patient.

### (6) Method of Producing the DENV4 VLP

In the sixth aspect, the present disclosure provides a method of producing the DENV4 VLP provided in the first aspect of the present disclosure, comprising culturing a cell which is expressing a gene coding for the chimeric DENV viral structural protein; and recovering the virus like particle from the cell culture.

Various host-vector systems may be used for expression of the virus like particle. Eukaryotic cells can be used for the method provided by the fourth aspect of the present disclosure. Examples of eukaryotic cells include, but are not limited to, insect cells (e.g., sf9 cells, H5 cells), yeast cells (e.g., S. cerevisiae) and mammalian cells (e.g., CHO cells, human embryonic kidney (HEK) 293F cells). Vector used for the method provided by the second aspect of the present disclosure comprises a nucleic acid molecule encoding the virus like particle to be expressed. Cells may be transfected with the vector using conventional methods (e.g., lipofection, electroporation). A skilled person can select culture medium or with DNA methyl transferase inhibitors and histone deacetylase inhibitors such as sodium butyrate, depending on cells employed. After the transfection, virus like particle can be produced in the cells and may be released into the culture supernatant. Virus like particle may be recovered from the cell culture suspension and purified using ultracentrifugation.

The DENV4 VLPs of the present disclosure do not replicate and therefore, have highly safe profiles.

### (7) a Cell Line Expressing the DENV4 VLP of the First Aspect.

In the seventh aspect, the present disclosure provides a cell line expressing the DENV4 VLP of the first aspect. The cell line may be obtained by transfecting Eukaryotic cells of a cell line by a vector discussed in the sixth aspect.

### (8) an Improved Method for Producing DENV VLP

In the eighth aspect, the present disclosure provides a method for producing dengue virus like particles comprising: culturing a cell transfected with a gene encoding at least one dengue virus viral structural proteins contained in the DENV VLP: adjusting the pH of the resulting cell culture suspension to a range from about 7 to about 9; and filtering the cell culture suspension to remove the cells and recover the DENV VLP released into the cell culture suspension. In the filtering step, the filter may be a depth filter which is commonly used in large scale manufacturing of proteins. As the depth filter, commercially available filters may be used. It was observed that DENV4 VLP in a cell culture suspension did not readily passed-through depth filters when the pH of the cell culture suspension was below 7, resulting in reduced filtration efficiency. In contrast, adjusting the pH to 7 or higher significantly improves passage through the filters, thereby mitigating the problem. These results indicate that maintaining the pH at 7 or higher during the process can enhance filtration performance. Itis expected that the same effect can be achieved with all DENV VLP including type 1-3 DENV VLP.

The pH may be adjusted using any suitable base, such as sodium hydroxide, potassium hydroxide, or ammonium hydroxide. In preferred embodiments, the pH is maintained in the range of about 7 to about 9 to optimize both product yield and process efficiency. The precise pH may be selected depending on the specific starting materials and reaction conditions, and can be adjusted to balance filtration performance with other process parameters.

As used herein, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive.

Unless specifically stated or obvious from context, as used herein, the terms “a”, “an”, and “the” are understood to be singular or plural.

When referring to pH, “about” includes variations attributable to measurement error and experimental conditions, typically within ±0.2 pH units.

The present disclosure will be described in detail with reference to the following examples, which, however, is not intended to limit the scope of the present disclosure.

### Example 1

### Preparation of a Cell Line Stable Expressing a DENV4 VLP

The old and new chimeric Dengue virus viral structural protein as shown in the lower panel of FIG. 2 was prepared. In the envelope region of the viral structural protein, amino acid Phe at position 108 of envelope protein of Dengue virus type 4 was altered to Ala (F108A).

Construct 1
(SEQ ID NO: 1)
MTSLCLMMILPATLAFHLTSRDGEPRMIVGKNERGKSLLFKTASGINMCT
LIAMDLGEMCDDTVTYKCPLIAEVEPEDIDCWCNLTSTWVTYGTCNQAGE
HRRDKRSVALAPHVGMGLDTRTQTWMSAEGAWRQVEKVETWALRHPGFTI
LALFLAHYIGTSLTQKVVIFILLMLVTPSMTMRCVGVGNRDFVEGLSGAT
WVDVVLEHGGCVTTMAKNKPTLDFELIKTTAKEVALLRTYCIEASISNIT
TATRCPTQGEPYLKEEQDQQYICRRDVVDRGWGNGCGLAGKGGVVTCAKF
SCSGKITGNLVQIENLEYTVVVTVHNGDTHAVGNDTSNHGVTATITPRSP
SVEVELPDYGELSLDCEPRSGIDENEMILMKMEKKTWLVHKQWFLDLPLP
WTAGADTSEVHWNHKERMVTFKVPHAKRQDVTVLGSQEGAMHSALTGATE
VDSGDGNHMFAGHLKCKVRMEKLRIKGMSYVMCTGSFKLEKEVAETQHGT
VLVQVKYEGTDAPCKIPFSSQDEKGVTQNGRLITANPIVTDKEKPVNIEA
EPPFGESYIVVGAGEKALKLSWFKKGSSIGKMFEATARGARRMAILGDTA
WDFGSIGGVFTSVGKLIHQIFGTAYGVLFSGVSWTMKIGIGILLTWLGLN
SRSTSLSMTCIAVGMVTLYLGVMVQA

The underlined amino acid corresponds to F108A.

Construct 1 is composed of the following parts. Positions of the amino acids shown below indicate the amino acid positions on SEQ ID NO: 1.

Signal sequence from capsid (DENV3) 1-15aa
(SEQ ID NO: 2)
pr (DENV3) 16-106 (SEQ ID NO: 3)
M(DENV3) 107-181aa (SEQ ID NO: 4)

Envelope region

E 182-218aa (SEQ ID NO: 5) is from DENV3
E 219-477aa (SEQ ID NO: 6) is from DENV4
E 478-676aa (SEQ ID NO: 7) is from DENV1

The amino acid sequence of the chimeric Dengue virus viral structural protein of the old DENV4 VLP is shown in SEQ ID NO: 8.

The 293F cells stably expressing the new DENV4 VLP was constructed in the following manner:

293F cells (ThermoFisher #11625019, Lot #236910) revived into 30 mL suspension media (293 SFM II, ThermoFisher #11686-029). 293F cells were passaged and expanded in shaker suspension culture (37° C., 5% CO2, 85% relative humidity, 125 rpm) for 7 days before conversion to adherent culture in adherent media (DMEM+10% FBS+1% pen/strep). 3 days after conversion to adherent culture, 5×10⁶ 293F cells were resuspended in 500 μL PBS and electroporated (4 mm cuvettes, 180V, 975 μF) with 5 μg linearized new chimeric DENV plasmid (endotoxin free, linearized via SapI) then plated onto a 10 cm dish in adherent media. Cells were then treated with 100 μg/mL hygromycin beginning at 72 h.p.t. until 21 days post-transfection, with media changes every 96-168 h. Surviving cells were counted and diluted to 9.6 cells/mL by limiting dilution before plating 0.1 mL per well onto 96-well plates. Single-cell clones were expanded for 28 days before VLP expression in the media was measured by dot blot, and clones with robust expression of new DENV4 VLP were gradually expanded. The clone selected was then re-adapted to serum-free media by a gradual reduction of FBS % every 48 h (10%, 5%, 2.5%, 1.25%, 0%) before transferring to suspension media in shaker suspension culture.

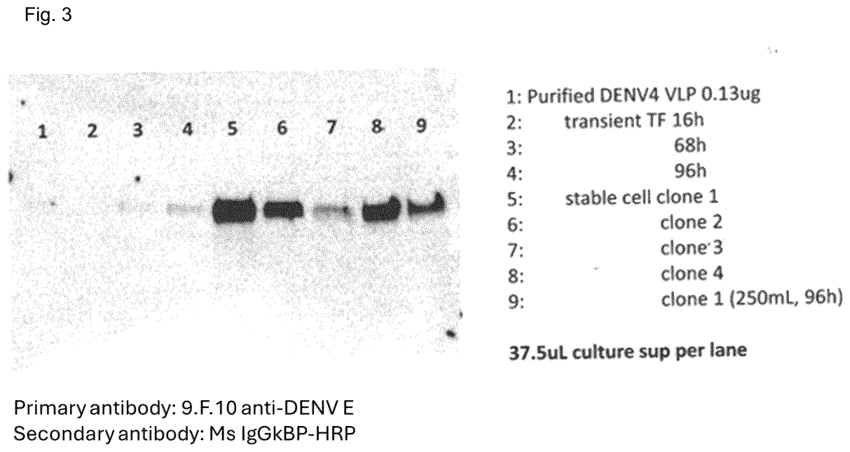

Aliquots of the serum-free adapted new chimeric DENV clone 1 were then cryopreserved (SFM+10% DMSO) at a concentration of 1×10⁷ cells/mL. These vials constituted the new DENV4 VLP-expressing 293F cells. Clonal expansion of new DENV4 VLP-expressing stable cell lines results in the robust release of the new DENV4 VLP into the culture supernatant (FIG. 3).

### Example 2

### Mouse Immunogenicity Study

A total of 21 female BALB/c mice (aged 9-10 weeks at time of first immunization) were assigned to three dosing groups (n=7 per group) and immunized a total of three times (days 1, 22 and 43) with one of three different test articles: 1 μg old DENV4 VLP. The amino acid sequence of the old DENV4 VLP (transient construct, which is disclosed in U.S. Pat. No. 10,098,943 as SEQ ID NO: 24)+adjuvant, 1 μg new (stable Construct 1)+adjuvant, or Sucrose Phosphate buffer (vehicle control). Each mixture of DENV4 VLP+adjuvant was prepared <4 h prior to immunization and stored at 2-8° C. until intramuscular administration of 50 μL doses containing the above test articles. Body weight was measured and serum samples collected by facial vein (for immunogenicity analysis) immediately prior to each immunization, with a terminal serum sample collection conducted at day 63 (21 days after third immunization). Immunogenicity in all 21 mice at each timepoint was measured by anti-DENV4 ELISA. ELISA Method: Nunc-Immuno 96-well plates (ThermoFisher #442404) were coated with 0.1 μg VLP/well overnight at 2-8° C. then blocked with Tris-buffered saline+0.05% Tween20 (TBST)+5% milk for 1 h at 20-25° C. before treatment with 100 μL serially diluted mouse serum samples (Dilutions ranged from 1:50 to 1:156,250) overnight at 2-8° C. Wells were then washed 5× with TBST before treatment with 100 μL HRP-conjugated Ms IgGK-BP at 400 ng/ml (Santa Cruz Biotechnology #516102) for 1 h at 20-25° C. Wells were then washed 5× with TBST, then total signal was visualized by 5 min treatment with 100 μL TMB Peroxidase (SeraCare #5120-0075) before stopping with 100 μL 2N H2SO4. Total antibody binding in each well was measured by 562 nm reading via Cytation7 plate reader (Omega BioTEK), and the endpoint dilution for each animal and timepoint was interpolated by least squares fit of a sigmoidal curve (OD=1.0 chosen for endpoint).

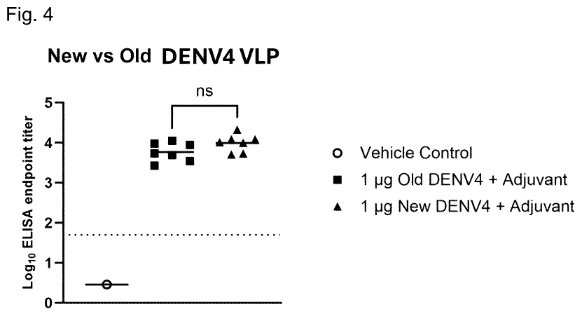

The DENV4 Immunogenicity conferred by immunization with 1 μg old DENV4 VLP (transiently-expressed VLP) vs 1 μg new DENV4 VLP (stably-expressed VLP, Construct 1) are shown in FIG. 4. The data indicates that new stable expressed new chimeric DENV VLP shows the comparable immunogenicity to old chimeric DENV VLP.

### Example 3

### Method

The 293F cells stably expressing the new DENV4 VLP in Example 1 were cultured in a triangular flask under shaking conditions at 37° C. with 8% CO₂ for 3 days (shaking speed: 130 rpm). Benzonase® Nuclease was added to the cell suspension to a final concentration of 1 U/mL, followed by incubation at 37° C. for 30 min. The resulting cell suspension was divided into two 200-mL portions, and subjected to one of the following treatments in order to remove the cells:

Treatment 1: the salt concentration of the cell suspension was increased with phosphate-buffered saline, followed by filtration using Supracap™ 50 depth filter capsule (Pall Corporation).

Treatment 2: the pH of the cell suspension was adjustment with 7.5% sodium bicarbonate solution, followed by depth filtration using Supracap™ 50 depth filter capsule (Pall Corporation).

The DENVLP-4 concentrations in the filtrates were measured by ELISA.

### Results

The results are shown in the table below. The DENV4 VLP concentration before treatment was 13.07 μg/mL. In the filtrate obtained after PBS addition and filtration was 0.24 μg/mL, whereas the concentration in the filtrate obtained after pH adjustment and filtration was 5.63 μg/mL. These results suggest that although DENV4 VLP does not readily pass through the depth filters commonly used in large-scale manufacturing, this can be mitigated by adjusting the pH.

TABLE 1

| Treatment | pH after treatment | DENVLP-4 Concentration |
|---|---|---|
| Before treatment | 6.51 | 13.07 μg/mL |
| Treatment 1 | 6.64 | 0.24 μg/mL |
| Treatment 2 | 7.01 | 5.63 μg/mL |

As shown in the table, maintaining the pH of the cell culture suspension at 7 or higher enhances the passage of component DENV4 VLP through depth filters. These results suggest that adjusting the pH of the cell culture suspension to the range of about 7 to about 9 is advantageous for improving filtration performance while maintaining product quality. The pH may be adjusted using any suitable base, such as sodium hydroxide or potassium hydroxide, and can be optimized depending on the reaction conditions and desired outcome.

### Example 4

### Tetravalent Composition

Tetravalent composition comprising DENV1 VLP, DENV2 VLP, DENV3 VLP and DENV4 VLP was prepared. The amino acid sequences of each VLPs are as follows

DENV1 VLP: SEQ ID NO: 9
DENV2 VLP: SEQ ID NO: 10
DENV3 VLP: SEQ ID NO: 11
DENV4 VLP: SEQ ID NO: 1

The amino acid sequences of DENV1-3 VLPs were shown in U.S. Pat. No. 10,098,943

DENV1-4 VLPs were prepared in the same manner as Example 1. To prepare a pharmaceutical composition which is a vaccine composition, 80 μg of each of the prepared particles was mixed with 1 ml of Sucrose Phosphate Solution, pH 7.2, Endotoxin Free (Teknova, SP buffer).

What is claimed is:

1. A dengue virus like particle specific for type 4 dengue virus (DENV4 VLP) comprising a chimeric dengue virus viral structural protein, wherein said chimeric dengue virus viral structural protein comprises prM and envelope regions and is a chimera of parts each derived from DENV1, DENV3 and DENV4.

2. The DENV4 VLP according to claim 1, wherein said dengue virus viral structural protein is a protein comprising an amino acid sequence represented by SEQ ID No: 1 or an amino acid sequence having at least 70%, 75%, 80%, 85%, 90%, 95% or 98% amino acid sequence identity to SEQ ID NO. 1.

3. An isolated nucleic acid molecule comprising a nucleotide sequence that encodes the dengue structural protein contained in the DENV4 VLP according to claim 1.

4. A vector comprising the nucleic acid molecule according to claim 3, wherein the vector optionally comprises an expression control sequence operably linked to the nucleic acid molecule.

5. A pharmaceutical composition comprising:
(a) the DENV4 VLP according to claim 1, the nucleic acid molecule encoding the DENV VLP, and/or the vector comprising the nucleic acid molecule; and
(b) a pharmaceutically acceptable carrier.

6. The pharmaceutical composition according to claim 5, further comprising dengue virus like particles specific for type 1, type 2 and type 3 dengue virus respectively (DENV1 VLP, DENV2 VLP and DENV3 VLP), isolated nucleic acid molecules, each of which encodes the dengue viral structural proteins contained in one of the VLPs, and/or vectors comprising the nucleic acid molecules, which optionally include an expression control sequence operably linked to the nucleic acid molecule.

7. A vaccine composition comprising the DENV4 VLP according to claim 1, the nucleic acid molecule encoding the DENV4 VLP, and/or the vector comprising the nucleic acid molecule.

8. The vaccine composition according to claim 7, further comprising dengue virus like particles specific for type 1, type 2 and type 3 dengue virus respectively (DENV1 VLP, DENV2 VLP and DENV3 VLP), isolated nucleic acid molecules, each of which encodes the dengue viral structural proteins contained in one of the VLPs, and/or vectors comprising the nucleic acid molecules, which optionally include an expression control sequence operably linked to the nucleic acid molecule.

9. A method of producing an antibody, comprising contacting the DENV4 VLP according to claim 1, the nucleic acid molecule encoding the DENV4 VLP, and/or the vector comprising the nucleic acid molecule to a mammal.

10. A method of treating or preventing a disease or condition caused by dengue virus infection, comprising administering an effective amount of the composition of claim 5 to a mammalian subject in need thereof.

11. A method for producing the DENV4 VLP according to claim 1, comprising culturing a cell transfected with a gene encoding at least one dengue virus viral structural proteins contained in the DENV4 VLP; and recovering the DENV4 VLP from the cell culture suspension.

12. A cell line expressing the DENV4 VLP which comprises an amino acid sequence represented by SEQ ID NO:1.

13. The cell line according to claim 12, wherein the cell line is a stable cell line.


SEQUENCE LISTING (11 sequences)

SEQ ID NO: 1 - protein, 676 aa, Dengue virus
MTSLCLMMILPATLAFHLTSRDGEPRMIVGKNERGKSLLFKTASGINMCTLIAMDLGEMCDDTVTYKCPLIAEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDFELIKTTAKEVALLRTYCIEASISNITTATRCPTQGEPYLKEEQDQQYICRRDVVDRGWGNGCGLAGKGGVVTCAKFSCSGKITGNLVQIENLEYTVVVTVHNGDTHAVGNDTSNHGVTATITPRSPSVEVELPDYGELSLDCEPRSGIDFNEMILMKMEKKTWLVHKQWFLDLPLPWTAGADTSEVHWNHKERMVTFKVPHAKRQDVTVLGSQEGAMHSALTGATEVDSGDGNHMFAGHLKCKVRMEKLRIKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 2 - protein, 15 aa, Dengue virus
MTSLCLMMILPATLA

SEQ ID NO: 3 - protein, 91 aa, Dengue virus
FHLTSRDGEPRMIVGKNERGKSLLFKTASGINMCTLIAMDLGEMCDDTVTYKCPLIAEVEPEDIDCWCNLTSTWVTYGTCNQAGEHRRDKR

SEQ ID NO: 4 - protein, 75 aa, Dengue virus
SVALAPHVGMGLDTRTQTWMSAEGAWRQVEKVETWALRHPGFTILALFLAHYIGTSLTQKVVIFILLMLVTPSMT

SEQ ID NO: 5 - protein, 37 aa, Dengue virus
MRCVGVGNRDFVEGLSGATWVDVVLEHGGCVTTMAKN

SEQ ID NO: 6 - protein, 259 aa, Dengue virus
KPTLDFELIKTTAKEVALLRTYCIEASISNITTATRCPTQGEPYLKEEQDQQYICRRDVVDRGWGNGCGLAGKGGVVTCAKFSCSGKITGNLVQIENLEYTVVVTVHNGDTHAVGNDTSNHGVTATITPRSPSVEVELPDYGELSLDCEPRSGIDFNEMILMKMEKKTWLVHKQWFLDLPLPWTAGADTSEVHWNHKERMVTFKVPHAKRQDVTVLGSQEGAMHSALTGATEVDSGDGNHMFAGHLKCKVRMEKLRIKG

SEQ ID NO: 7 - protein, 199 aa, Dengue virus
MSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 8 - protein, 676 aa, Dengue virus
MSTVTLLCLIPTVMAFHLSTRDGEPLMIVAKHERGRPLLFKTTEGINKCTLIAMDLGEMCEDTVTYKCPLLVNTEPEDIDCWCNLTSTWVMYGTCTQNGERRREKRSVALTPHSGMGLETRAETWMSSEGAWKHAQRVESWILRNPGFALLAGFMAYMIGQTGIQRTVFFVLMMLVAPSYGMRCIGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLAGKGGVVTCAKFSCSGKITGNLVQIENLEYTVVVTVHNGDTHAVGNDTSNHGVTATITPRSPSVEVELPDYGELSLDCEPRSGIDFNEMILMKMEKKTWLVHKQWFLDLPLPWTAGADTSEVHWNHKERMVTFKVPHAKRQDVTVLGSQEGAMHSALTGATEVDSGDGNHMFAGHLKCKVRMEKLRIKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 9 - protein, 676 aa, Dengue virus
MSVTMLLMLLPTALAFHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLAGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 10 - protein, 676 aa, Dengue virus
MTAGMIIMLIPTVMAFHLTTRNGEPHMIVSRQEKGKSLLFKTKDGTNMCTLMAMDLGELCEDTITYKCPFLKQNEPEDIDCWCNSTSTWVTYGTCTTTGEHRREKRSVALVPHVGMGLETRTETWMSSEGAWKHAQRIETWILRHPGFTIMAAILAYTIGTTHFQRVLIFILLTAIAPSMTMRCIGISNRDFVEGVSGGSWVDIVLEHGSCVTTMAKNKPTLDFELIKTEAKQPATLRKYCIEAKLTNTTTDSRCPTQGEPTLNEEQDKRFVCKHSMVDRGWGNGCGLAGKGGIVTCAMFTCKKNMEGKIVQPENLEYTVVITPHSGEEHAVGNDTGKHGKEVKITPQSSITEAELTGYGTVTMECSPRTGLDFNEMVLLQMKDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 11 - protein, 674 aa, Dengue virus
MTSLCLMMILPATLAFHLTSRDGEPRMIVGKNERGKSLLFKTASGINMCTLIAMDLGEMCDDTVTYKCPLIAEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLAGKGSLVTCAKFQCLEPIEGKVVQHENLKYTVIITVHTGDQHQVGNDTQGVTVEITPQASTVEAILPEYGTLGLECSPRTGLDFNEMILLTMKNKAWMVHRQWFFDLPLPWTSGATTEAPTWNRKELLVTFKNAHAKKQEVVVLGSQEGAMHTALTGATEIQNSGGTSIFAGHLKCRLKMDKLELKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA